SEQUENCE LISTING (4 sequences)

SEQ ID NO: 1 - DNA, 9555 bp, Artificial
  pNOV2145
gatccaccggtcgccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgagcggccctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaatggcgcgccggtaccagcttgcatgcctgcaggtcgactctagaggatcctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtaggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccatgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctaaatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgtacgtagcactagtgcgcgatcgcttaattaagcggcgcgcctaaagcttctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaag

SEQ ID NO: 2 - DNA, 9546 bp, Artificial
  pNOV2147
ggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccatgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctaaatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgtacgtagcactagtgcgcgatcgcttaattaagcggcgcgcctaaagcttctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaggatccaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgagagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaatggcgcgccggtaccagcttgcatgcctgcaggtcgactctagaggatcctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagta

SEQ ID NO: 3 - DNA, 10604 bp, Artificial
  pBSC11234
cgcgcctaaagcttgcatgcctgcaggtcgactctagaggatcctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtaggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccatgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctaaatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgtacgtagcactagtgcgcgatcgcttaattaagcggcgcgcctgcaggcggccgcacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcgatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagaagacaaaacacaatgggagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttcatcatcaagaaccagttcaatatcctagtacaccgtattaaagaatttaagatatactccaccggatccaccatggccaagctagttttttccctttgttttctgcttttcagtggctgctgcttcgctgagattttcggcaagaccttccgcgagggccgcttcgtgctcaaggagaagaacttcaccgtggagttcgccgtggagaagatccacctcggctggaagatatcgggccgcgtgaagggctcgccgggccgcctcgaggtgctccgcaccaaggccccggagaaggtgctcgtgaacaactggcagtcctggggcccgtgccgcgtggtggacgccttctccttcaagccgccggagatcgacccgaactggcgctacaccgcatccgtggtgccggacgtgctcgagcgcaacctgcagtccgactacttcgtggccgaggagggcaaggtgtacggcttcctctcctccaagatcgcccacccgttcttcgcggtggaggacggcgagctggtggcctacctcgagtacttcgacgtggagttcgacgacttcgtgccgctggagccgctcgtggtgctcgaggacccgaacaccccgctcctcctcgagaagtacgccgagctggtgggcatggagaacaacgcccgggtgccgaagcacacgccgaccggctggtgctcctggtatcactacttcctcgacctcacctgggaggagaccctcaagaacctcaagctcgccaagaacttcccgttcgaggtgttccagatcgacgacgcctacgagaaggacatcggcgactggctcgtgacccgcggcgacttcccgtccgtggaggagatggccaaggtgatcgccgagaacggcttcatccccggcatctggaccgccccgttctccgtgtccgagactagtgacgtgttcaacgagcacccggactgggtggtgaaggagaacggcgagccgaagatggcctaccgcaactggaacaagaagatttacgccctcgacctctccaaggacgaggtgctcaactggctcttcgacctcttctcctccctccgcaagatgggctaccgctacttcaagatcgacttcctcttcgcgggcgccgtgccgggggagcgcaagaagaacatcaccccgatccaggccttccgcaagggcatcgagaccatccgcaaggccgtgggggaggactccttcatcctcggctgcggctcccccctcctcccggccgtgggctgcgtggatggcatgcgcatcggcccggacaccgccccgttctggggagagcacatcgaggacaacggcgccccggcggcccgctgggccctccgcaacgccatcacccgctacttcatgcacgaccgcttctggctcaacgacccggactgcctcatcctccgcgaggagaagaccgacctcacccagaaggagaaggagctgtactcctacacctgcggcgttctagacaacatgatcatcgagtccgacgacctctccctcgtgcgcgaccacggcaagaaggtgctcaaggagaccctcgagctgctcgggggcaggccgcgcgtgcagaacatcatgtccgaggacctccgctacgagatcgtgtcctcgggcaccctctccggcaacgtgaagatcgtggtggacctcaactcccgcgagtaccacctcgagaaggagggcaagtcctccctcaagaagcgcgtggtgaagcgggaggacggcaggaacttctacttctacgaggagggcgagcgcgagtgaaagcttgacgtcactagtgcgatcgcgctagccatggccggcctaggcgcccgggagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcctcgagtctagacctgcagg

SEQ ID NO: 4 - DNA, 8757 bp, Artificial
  pBSC11369
aagcttctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaggatccaccggtcgccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgctctagatcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggggaaatttaccggtgccgaatttccccgatccagcttctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaggatccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagggatcccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcggcccgaattcactggccgtcgttttacaacgtcgtgactgggaaaaccctggcgttacccaacttaatcgccttgcagcacatccccctttcgccaggggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcggtac

(19) United States
(12) Patent Application Publication
(10) Pub. No.: US 2006/0260012 A1
(43) Pub. Date: Nov. 16, 2006

(54) Method of transforming soybean

(75) Inventor: Rafiqul Khan, RESEARCH TRIANGLE PARK, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: SYNGENTA PARTICIPATIONS AG

(21) Appl. No.: 10/517,039

(22) PCT Filed: Jun. 17, 2003
(86) PCT No.: PCT/US03/19212

§ 371 Date: Dec. 7, 2004

Related U.S. Application Data
(60) Provisional application No. 60/390,562, filed on Jun. 22, 2002.

(51) Int. Cl. A01H 5/00; C12N 15/82; C12N 5/04

(52) U.S. Cl. 800/287; 800/312; 435/468; 435/415

(57) ABSTRACT

The present disclosure provides methods for Agrobacterium-mediated transformation of soybean cells or tissue and regeneration of the transformed cells or tissue into transformed plants. The methods may be used for transforming many soybean cultivars.

### CROSS REFERENCE TO RELATED APPLICATIONS

This application claims the benefit of U.S. Provisional Application Ser. No. 60/390,562, filed Jun. 22, 2002, the entire contents of which is hereby incorporated by reference.

### FIELD OF THE INVENTION

The invention relates generally to methods for plant transformation and, more particularly, to methods for transforming soybean cells or tissues. The invention also relates to methods for regenerating transgenic soybean plants from transformed soybean cells or tissues. The invention also relates to transgenic soybean plants and seeds obtained by such methods.

### BACKGROUND

Soybean is a major food and feed source that is grown on more acres worldwide than any other dicotyledonous crop. It is reportedly grown on more than 50 million hectares. Unfortunately, only a few plant introductions have given rise to the major cultivars grown in the United States and, as a consequence, this narrow germplasm base has limited soybean breeding potential. The limited genetic base in domestic soybean varieties has limited the power of traditional breeding methods to develop varieties with improved or value-added traits.

Hence, the use of genetic engineering techniques to modify soybean can facilitate the development of new varieties with, for example, traits such as herbicide resistance, disease resistance (such as virus resistance, for example), and seed quality improvement in a manner that has been unattainable by traditional breeding methods or tissue-culture induced variation.

The development of an efficient transformation system is necessary for the analysis of gene expression in plants. The requirements for such a system include a proper target plant tissue that will allow efficient plant regeneration, a gene delivery vehicle that delivers foreign DNA efficiently into the target plant cells, and an effective method for selecting transformed cells. In genetic transformation of dicotyledonous specics, transformation systems utilizing the bacterium Agrobacterium tumefaciens have been frequently used as vehicles for gene delivery. The preferred target tissues for Agrobacterium-mediated transformation presently include cotyledons, leaf tissues, and hypocotyls. High velocity microprojectile bombardment offers an alternative method for gene delivery into dicotyledonous plants.

Agrobacterium-mediated gene delivery in soybean has been far from routine. In reports that have been available to the public, meristems and cotyledon tissues have been frequently mentioned as targets for use in Agrobacterium-mediated gene delivery. However, reliable and efficient transformation and regeneration from these two explant sources are often not accomplished.

U.S. Pat. No. 5,169,770 and 5,376,543 to Chee et al. discuss a non-tissue culture method of transforming soybeans to produce transgenic plants, wherein seeds are germinated and meristematic or mesocotyl cell tissues are inoculated with bacterial cells, specifically Agrobacterium strains, which, through infection, transfer DNA into the explants. This method depends on the growth of preformed shoots.

Parrott W. A. et al. (1989), “Recovery of primary transformants of soybean,” Plant Cell Reports 7:615-617, report recovery of soybean transformants from immature cotyledon tissue after co-cultivation with Agrobacterium. However, the regenerated plants were chimeric, and the transgenes were not transmitted to the progeny.

U.S. Pat. No. 5,416,011 (to Hinchee et al.) discusses utilizing a cotyledon explant, which requires removal of the hypocotyl, saving and separating the cotyledons and inserting a chimeric gene by inoculation with Agrobacterium tumefaciens vectors containing the desired gene.

Yan B. et al. (2000), “Agrobacterium tumefaciens—mediated transformation of soybean using immature zygotic cotyledon explants,” Plant Cell Reports 19:1090-1097, report an overall 0.03% transformation frequency in Agrobacterium-mediated transformation in soybean with immature cotyledons.

U.S. Pat. No. 6,384,301 to Martinell et al. describes Agrobacterium-mediated gene delivery into cells in the meristem of an isolated soybean embryonic axis. Their method does not involve a callus-phase tissue culture.

From the work described above, it is clear that the goal of establishing a reliable soybean transformation system is seldom accomplished by the workers involved when meristems and cotyledon tissues are used as source explants for Agrobacterium-mediated gene delivery. Therefore, there is a need to continue to exploit new methodology, including new source explants, in order to develop a more efficient soybean transformation system.

It has been demonstrated in soybean tissue culture that plant regeneration may be achieved from epicotyl tissues and primary leaf tissues. However, to-date, no successful transformation has been reported in soybean using these two explant sources as targets for gene delivery.

Wright M. S. et al. (1987) “Initiation and propagation of Glycine max L. Merr.: Plants from tissue-cultured epicotyls,” Plant Cell Reports 8:83-90, describes successful initiation and proliferation of shoots from epicotyl tissue of soybean. Explanted epicotyls were induced to form shoots in Schenk and Hildebrandt medium containing 20 μM kinetin for 5 weeks. Shoot proliferation was maintained on N6 medium containing 2.1 nM picloram and 0.1 μM benzyladenine.

Wright M. S. et al. (1987) “Regeneration of soybean (Glycine max L. Merr.) from cultured primary leaf tissue,” Plant Cell Reports 6:83-89, describes a reproducible method for regeneration of plants from primary leaf tissue of 27 varieties of soybean They found that while 2,4,5-trichlorophenoxyacetic acid was demonstrated to be essential for regeneration, addition of benzyadenine (BA) was found to enhance regeneration.

Rajasckaren K. et al. (1997) “Somatic embryogenesis from cultured epicotyls and primary leaves of soybean (Glycine max L. Merr),” In Vitro Cellular & Developmental Biology 33(2):88-91, describes regeneration of several varieties of soybean by somatic embryogenesis from cultured epicotyls and primary leaf tissues of immature seeds from greenhouse grown plants. They found that somatic embryogenesis was induced from epicotyls and primary leaves when cotyledon halves with the intact zygotic embryo axes were cultured on Murashige and Skoog (MS) medium supplemented with 46.2 μM 2,4-D. In the absence of being cultured with the cotyledon halves, no embryogenesis was observed from isolated axes, epicotyls or primary leaves. Rapid multiplication of shoot tips from germinating somatic embryos was achieved on Cheng's basal medium containing 11.3 μM benzyladenine.

### SUMMARY

The present invention provides a method for transforming soybean cells and regeneration of the transformed cells into transformed plants. The method may be used for transforming many soybean cultivars.

The invention provides a novel soybean explant that enables Agrobacterium tumefaciens-mediated gene delivery into soybean cells with high efficiency.

In particular, the invention provides a method for transforming soybean cells or tissue, the method comprising:
(a) preparing an explant from a soybean seed by:
   (i) removing all or a part of the hypocotyl from said seed;
   (ii) removing one cotyledon along with its adjacent axillary bud from the seed, and leaving one cotyledon with the epicotyl and primary leaves attached thereto;
   (iii) removing a portion of a primary leaf from the remaining cotyledon, thereby generating a primary leaf base; and
(b) co-cultivating the explant with Agrobacterium comprising a nucleic acid of interest to be incorporated into the genome of the soybean cells.

In additional embodiments, the method further includes one or more of the following: inducing shoot formation from the primary leaf base and the adjacent epicotyl; cultivating the shoot in a medium containing a selection agent; selecting a transformed shoot; and regenerating a transformed plant from the transformed shoot.

In a further embodiment, the invention provides a method for producing a stably transformed soybean plant, the method comprising:
(a) preparing an explant from a soybean seed by:
   (i) removing all or a part of the hypocotyl from said seed;
   (ii) removing one cotyledon along with its adjacent axillary bud from the seed, and leaving one cotyledon with the epicotyl and primary leaves attached thereto;
   (ii) removing a portion of a primary leaf from the epicotyl, thereby generating at least one primary leaf base;
(b) co-cultivating the explant with Agrobacterium comprising a nucleic acid of interest to be incorporated into the genome of the soybean cells;
(c) inducing shoot formation from the primary leaf base area;
(d) cultivating a formed shoot in a medium containing a selection agent,;
(e) selecting a transformed shoot; and
(f) regenerating a selected transformed shoot into a soybean plant.

In another embodiment, a portion of each of the primary leaves of the explant generated in (a)(ii) is removed, thereby generating a pair of primary leaf bases.

The method of the invention may be employed to introduce any desired nucleic acid into a soybean cell. In one embodiment of the invention, the nucleic acid comprises a gene that would express a desirable agronomic trait in soybean.

In another embodiment of the invention, the nucleic acid comprises a phosphomannose isomerase gene, which is used as a selectable marker gene.

In an additional embodiment of the invention, the co-cultivating of the explant with Agrobacterium is carried out in the presence of mannose.

Both mature and immature seeds may be employed to generate the explant used in the present invention.

### BRIEF DESCRIPTION OF THE FIGURES

FIG. 1 shows a map of plasmid pNOV2105.

FIG. 2 shows a map of plasmid pNOV2145.

FIG. 3 shows a map of plasmid pNOV2147.

FIG. 4 shows an exemplary process for preparing a soybean explant. Panel A depicts a soybean seed embryo in which a part of the hypocotyl is removed. Panel B depicts the soybean explant from Panel A in which one cotyledon is removed along with its adjacent axillary bud. Panel C depicts the soybean explant from Panel B after removal of the two primary leaves, generating a break point at the base of each primary leaf.

FIG. 5 shows a map of plasmid pBSC11234.

FIG. 6 shows a map of plasmid pBSC11369.

### DETAILED DESCRIPTION

The present invention will now be described more fully hereinafter with reference to the accompanying figures, in which various embodiments of the invention are described. This invention may, however, be embodied in different forms and should not be construed as limited to the embodiments set forth herein. Rather, these embodiments are provided so that this disclosure will be thorough and complete and will fully convey the scope of the invention to those skilled in the art. The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs.

Except as otherwise indicated, standard methods may be used for the production of cloned genes, expression cassettes, vectors (e.g., plasmids), proteins and protein fragments, and transformed cells and plants according to the present invention. Except as otherwise indicated, standard methods may be used for the production of cloned genes, expression cassettes, vectors (e.g., plasmids), proteins and protein fragments according to the present invention. Such techniques are known to those skilled in the art. See e.g., J. Sambrook et al., Molecular Cloning: A Laboratory Manual Second Edition (Cold Spring Harbor Laboratory, Cold Spring Harbor, N.Y., 1989), and F. M. Ausubel et al., Current Protocols In Molecular Biology (Green Publishing Associates, Inc. and Wiley-Interscience, New York, 1991); J. Draper et al., eds., Plant Genetic Transformation And Gene Expression: A Laboratory Manual, (Blackwell Scientific Publications, 1988); and S. B. Gelvin & R. A. Schilperoort, eds., Introduction, Expression, And Analysis Of Gene Production In Plants.

The present invention is drawn to methods and compositions for the stable transformation of soybean with nucleic acid sequences of interest and the regeneration of transgenic soybean plants.

The methods of the invention may be employed to express any nucleic acid of interest in soybean plants. A gene of interest may be, for example, a gene for herbicide resistance, disease resistance, or insect/pest resistance, or is a selectable or scorable marker, and comprises a plant-operable promoter, a coding region, and a 3′ terminator region. Herbicide resistance genes include the AHAS gene for resistance to imidazolinone or sulfonyl urea herbicides, the pat or bar gene for resistance to bialaphos or glufosinate, the EPSP synthase gene for resistance to glyphosate, etc. Disease resistance genes include genes for antibiotic synthetic enzymes, e.g., for pyrrolnitrin synthetic enzymes, plant derived resistance genes, and the like. Insect resistance genes include genes for insecticidal proteins from Bacillus thuringiensis. Genes of interest may also encode enzymes involved in biochemical pathways, the expression of which alters a trait that is important in food, feed, nutraceutical, and/or pharmaceutical production. The gene of interest may be located on a plasmid. A plasmid suitable for use in the present invention may comprise more than one gene of interest and/or the Agrobacterium may comprise different plasmids having different genes of interest.

The present invention provides a method for the transformation of varieties of soybean, including Glycine max. The method is based on Agrobacterium-mediated delivery of a desired gene into a soybean cell followed by regeneration of transformed cell(s) into a transformed soybean plant. The methods of the invention are cultivar independent.

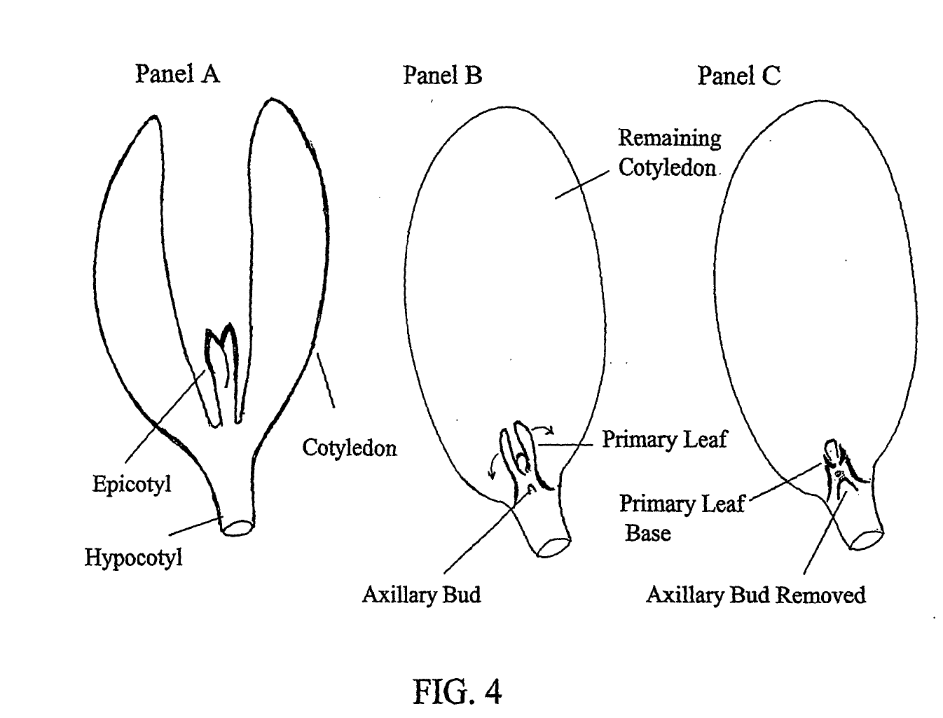

In one embodiment of the invention, an explant is prepared by germinating a soybean mature seed or immature seed collected from a greenhouse grown plant in a seed germination medium for a period of time, removing seed coat and, subsequently, a cotyledon from said mature seed or immature seed. In a preferred embodiment of the invention, a portion of the exposed primary leaves is then removed, thereby creating a break point at the primary leaf base (FIG. 4). Agrobacterium-mediated gene delivery is made into the cells at the primary leaf base or in the area of the primary leaf break point. Adventitious shoots are induced from the primary leaf base area of the epicotyl. This induction is achieved by removing pre-existing meristems (i.e., primary, secondary, and axillary meristems) and subjecting the explant to a shoot induction medium containing appropriate growth regulators, The shoot induction process facilitates the development or regeneration of transformed shoots from the targeted primary leaf base cells.

Transformed soybean cells are cultured in the presence of a selection agent. Preferably, the cells are transformed with a phosphomannose isomerase (PMI gene, and the transformed cells are cultivated in the presence of mannose. In a medium that contains mannose as a selection agent, soybean cells transformed with a PMI gene have a growth advantage over those that are not so transformed.

The time required for regenerating a transformed soybean plant using the method described in this invention is significantly reduced compared to other Agrobacterium-mediated transformation protocols that are reported in the literature. A rooted transformed soybean shoot may be produced 8 to 12 weeks from the initiation of a transformation experiment. A foreign genetic construct, or transgene, to be inserted into the soybean genome is created in vitro by normal techniques of recombinant DNA manipulations. The construct may be comprised of any heterologous nucleic acid. The genetic construct is transformed into the Agrobacterium strain for delivery into the soybean cells. The Agrobacterium is non-oneogenic, and several such strains are now widely available. The Agrobacterium is preferably selected from A. tumefaciens and A. rhizogenes.

The foreign genetic construct preferably comprises a selectable marker gene. The preferred selectable marker gene is a phosphomannose isomerase gene. Other suitable selectable marker genes include, but are not limited to, genes encoding: neomycin. phosphotransferase II (Fraley et al., CRC Critical Reviews in Plant Science 4, 1 (1986)); cyanamide hydratase (Maier-Greiner et al., Proc. Natl. Acad. Sci. USA 88, 4250 (1991)); aspartate kinase; dihydrodipicolinate synthase (Perl et al., BioTechnology 11, 715 (1993)); bar gene (Toki et al., Plant Physiol. 100, 1503 (1992); Meagher et al., Crop Sci. 36, 1367 (1996)); tryptophane decarboxylase (Goddijn et al., Plant Mol. Biol. 22, 907 (1993)); neomycin phosphotransferase (NEO; Southern et al., J. Mol. Appl. Gen. 1, 327 (1982)); hygromycin phosphotransferase (HPT or HYG; Shimizu et al., Mol. Cell. Biol. 6, 1074 (1986)); dihydrofolate reductase (DHFR); phosphinothricin acetyltransferase (DeBlock et al., EMBO J. 6, 2513 (1987)); 2,2- dichloropropionic acid dehalogenase (Buchanan-Wollatron et al., J. Cell. Biochem. 13D, 330 (1989)); acetohydroxyacid .synthase (U.S. Pat. No. 4,761,373 to Anderson et al.; Haughn et al., Mol. Gen. Genet. 221, 266 (1988)); 5-enolpyruvyl-shikimate-phosphate synthase (aroA; Comai et al., Nature 317, 741 (1985)); haloaryinitrilase (WO 87/04181 to Stalker et al.), acetyl-coenzyme A carboxylase (Parker et al., Plant Physiol. 92, 1220 (1990)); dihydropteroate synthase (sulI; Guerineau et al., Plant Mol. Biol. 15, 127 (1990)); and 32 kDa photosystem II polypeptide (psb A; Hirschberg et al., Science 222, 1346 (1983)).

Also included are genes encoding resistance to chloramphenicol (Herrera-Estrella et al., EMBO J. 2, 987 (1983)); methotrexate (Herrera-Estrella et al., Nature 303, 209 (1983); Meijer et al., Plant Mol. Biol. 16, 807 (1991)); hygromycin (Waldron et al., Plant Mol. Biol. 5, 103 (1985); Zhijian et al., Plant Science 108, 219 (1995); Meijer et al., Plant Mol. Bio. 16, 807 (1991)); streptomycin (Jones et al., Mol. Gen. Genet. 210, 86 (1987)); spectinomycin (Bretagne- Sagnard et al., Transgenic Res. 5, 131 (1996)); bleomycin (Hille et al., Plant Mol. Biol. 7, 171 (1986)); sulfonamide (Guerineau et al., Plant Mol. Bio. 15, 127 (1990); bromoxynil (Stalker et al., Science 242, 419 (1988)); 2,4-D (Streber et al., Biol/Technology 7, 811 (1989)); phosphinothricin (DeBlock et al., EMBO J. 6, 2513 (1987)); spectinomycin (Bretagne-Sagnard and Chupeau, Transgenic Research 5, 131 (1996)).

In one embodiment, the starting material for the transformation process is a soybean mature seed. In another embodiment, the starting material can be a soybean immature seed from a growing soybean plant. The seed is placed on a germination medium and permitted to germinate for a period of 6-24 hours, preferably for about 6-14 hours, and more preferably for about 8-12 hours. Seeds may also be allowed to germinate for a longer period of time, for example, from 2 to 5 days, if desired.

The seed coat and hypocotyl of the germinating seed is removed. One cotyledon along with its adjacent axillary shoot bud is also removed. Afterwards, the primary leaves are substantially removed, thereby creating an explant comprising the primary leaf base, epicotyl to which the leaf base is attached, and a cotyledon to which the epicotyl is attached. Substantially removed means removal of a major portion of primary leaf tissue.

For Agrobacterium-mediated gene transfer, wounding of the plant tissue is known to facilitate gene transfer. Therefore it is preferred, but not necessary, that a wound is created at the leaf base region.

The explant, prepared as described above, is then immersed into an Agrobacterium cell suspension for a few minutes to a few hours, typically about 0.5-3 hours, and preferably 1-2 hours. Excessive Agrobacterium cell suspension is removed and the remaining Agrobacterium are permitted to co-cultivate with the explant on a co-cultivation medium for several days, typically two to five days, and preferably three to four days, under 16h light/8h dark conditions at a temperature of about 22° C.±2° C.

After co-cultivation, the explant is transferred to a medium (or a series of media) conducive to shoot development and selection of transformed cells, for 8-12 weeks. Such a medium (or media) generally contains a shoot-inducing hormone as well as a selection agent. The regeneration media used in the examples below contain mannose, as the selection agent, as well as benzylaminopurine (BAP), a shoot-inducing hormone. The term hormone also includes cell growth regulating compounds that induce shoot formation, including, but not limited to, auxins (such as, e.g., IAA, NAA, and indole butyric acid (IBA)), cytokinins (such as, e.g., thidiazuron, kinetin, and isopentenyl adenine), and/or gibberellic acids (GA₃).

When shoots reach about 2 cm and with full trifoliate leaf formation, shoots are separated from the explant and placed on a rooting medium to induce root formation. Preferably, the rooting medium also contains a selection agent to further help identify potential transformed shoots. Root formation takes approximately 1-2 weeks, following which the plants can be transferred to soil and grown to full maturity.

Transgenic plants comprising a heterologous nucleic acid (i.e., comprising cells or tissues transformed in accordance with the methods described herein), as well as the seeds and progeny produced by the transgenic plants, are an additional aspect of the present invention. Procedures for cultivating transformed cells to useful cultivars are known to those skilled in the art. Techniques are known for the in vitro culture of plant tissue, and in a number of cases, for regeneration into whole plants. A further aspect of the invention is transgenic plant tissue, plants, or seeds containing the nucleic acids described above. In a preferred embodiment, transformed plants produced using the methods described herein are not chimeric, or only a small proportion of transformed plants is chimeric. This is preferably achieved by extending the period of high cytokinin treatment or by increasing the stringency of mannose selection, or both.

Thus, the transformed cells of the present invention, identified by selection or screening and cultured in an appropriate medium that supports regeneration as provided herein, may then be allowed to mature into plants. Plants are preferably matured either in a growth chamber or greenhouse. Plants are regenerated from about 2-6 weeks after a transformant is identified, depending on the initial tissue. During regeneration, cells may be grown on solid media in tissue culture vessels. Illustrative embodiments of such vessels are petri dishes and Plant Con®s. After the regenerating plants have reached the stage of shoot and root development, they may be transferred to a greenhouse for further growth and testing. As provided above, seeds and progeny plants of the regenerated plants are an aspect of the present invention. Accordingly, the term “seeds” is meant to encompass seeds of the transformed plant, as well as seeds produced from the progeny of the transformed plants. Plants of the present invention include not only the transformed and regenerated plants, but also progeny of transformed and regenerated plants produced by the methods described herein.

Plants produced by the described methods may be screened for successful transformation by standard methods described above. Seeds and progeny plants of regenerated plants of the present invention may be continuously screened and selected for the continued presence of the transgenic and integrated nucleic acid sequence in order to develop improved plant and seed lines, which are another aspect of the present invention. Desirable transgenic nucleic acid sequences may thus be moved (i.e., introgressed or inbred) into other genetic lines such as certain elite or commercially valuable lines or varieties. Methods of introgressing desirable nucleic acid sequences into genetic plant lines may be carried out by a variety of techniques known in the art, including by classical breeding, protoplast fusion, nuclear transfer and chromosome transfer. Breeding approaches and techniques are known in the art, and are set forth in, for example, J. R. Welsh, Fundamentals of Plant Genetics and Breeding (John Wiley and Sons, New York, (1981)); Crop Breeding (1). R. Wood, ed., American Society of Agronomy, Madison, Wis., (1983)); O. Mayo, The Theory of Plant Breeding, Second Edition (Clarendon Press, Oxford, England (1987)); and Wricke and Weber, Quantitative Genetics and Selection Plant Breeding (Walter de Gruyter and Co., Berlin (1986)). Using these and other techniques in the art, transgenic plants and inbred lines obtained according to the present invention may be used to produce commercially valuable hybrid plants and crops, which hybrids are also an aspect of the present invention.

The foregoing is illustrative of the various embodiments of the present invention and is not to be construed as limiting thereof.

The invention will be further described by the following examples, which are not intended to limit the scope of the invention in any manner.

### EXAMPLE 1

Transformation Vectors

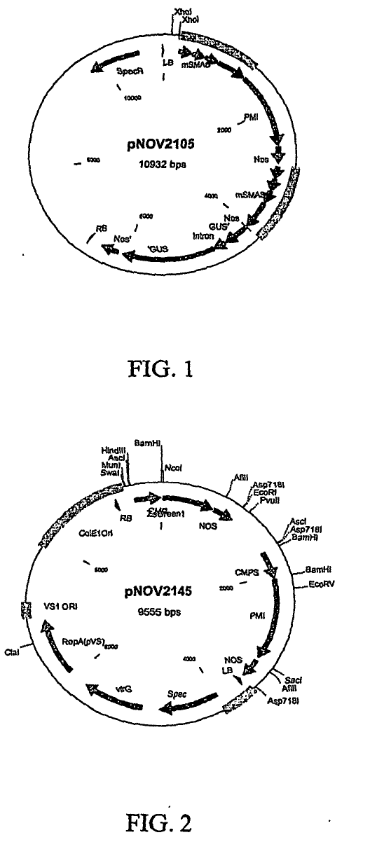

The plasmid pNOV2105 (FIG. 1) is a modification of p Victor, which is disclosed and described in WO 97/04112 in that the 35S promoter is replaced with a SMAS promoter, the 35S terminator is replaced with the Nos terminator, and an additional SMAS promoter is inserted upstream of the GUSintronGUS sequence, which is flanked on its 3′ end by a Nos terminator. pNOV2105 employed in the methods described herein does not contain the multicloning site that is found in p Victor. However, it is well within the skill in the art to add such a cloning site, if desired.

pNOV2105 (FIG. 1) is a vector for Agrobacterium-mediated plant transformation and contains the Ti right and left border sequences from the nopaline type pTiT37 plasmid (Yadav et al. 1982 Proc Natl Acad Sci 79:6322-6326) flanking the genes phosphomannose isomerase (PM) and beta-glucoronidase (GUS).

For replication and maintenance in E. coli, the plasmid contains the origin of replication from the E. coli plasmid pUC19 (pUC19ori) (Yanish-Perron et al. 1985 Gene 33:103-119), and for replication and maintenance in Agrobacterium tumefaciens the plasmid further contains the origin of replication from the Pseudomonas plasmid pVS1 (pVSlori) (Itoh et al. 1984 Plasmid 11:206-220; Itoh and Haas 1985 Gene 36:27-36). For selection in E. coli and Agrobacterium tumefaciens, the plasmid contains the spectinomycin/streptomycin resistance gene (spec/strep) from the transposon Tn7 encoding the enzyme 3″(9)-O-nucleotidyltransferase (Fling et al. 1985 Nucleic Acids Res 19:7095-7106). The spec/strep resistance gene is fused to the tac promoter (see, e.g., Amann et al. 1983 Gene 25(203):167-78) for efficient expression in the bacterium.

The T-DNA segment between the right and left border harbors the following genes, which are the only genes transferred to the soybean plant via the Agrobacterium tumefaciens-mediated transformation.

GUSintronGUS

beta-glucuronidase (GUS): This segment next to the right border contains the beta-glucuronidase gene (GUS) from E. coli with an intron in the coding region to prevent translation by Agrobacterium fused to the SMAS promoter and Nos terminator. The GUSintronGUS gene was isolated from plasmid pBISN1. (Narasimhulu et al. 1986 Early transcription of Agrobacterium DNA in tobacco and maize, Plant Cell 8:873-866).

phosphomannose isomerase (PMI): This segment next to the left border is the mannose-6-phosphate isomerases gene from E. coli (Miles and Guest 1984, Gene 32:41-48 ) fused to the SMAS promoter (Ni M, Cui D, Einstein J, Narasimhulu S, Vergara C E, Gelvin S B (1995) and Nos terminator. The phosphomannose isomerase gene is used as a selection marker to select transgenic shoots on media containing D-mannose as the carbon source.

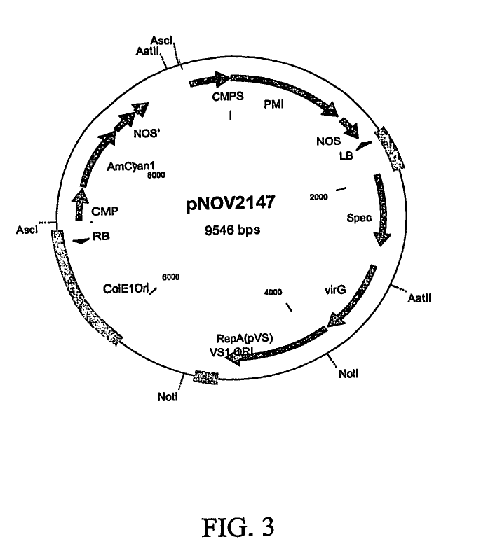

The components and sequence of pNOV2145 (FIG. 2) are set forth in SEQ ID NO:1. The components and sequence of pNOV2147 (FIG. 3) are set forth in SEQ ID NO:2.

### EXAMPLE 2

Transformation and Regeneration

Mature dried soybean seeds (Var. S42 H1) were surface sterlized by releasing chlorine gas inside a desiccator. Seeds were kept in petri plates and chlorine gas was produced by pouring 100 ml of Clorox® into a beaker and slowly adding 8 ml of concentrated HCl. Seeds were sterilized by at least two gas release treatments each lasting for 8-18 hours.

Sterilized seeds (approximately 15-20 seeds per plate) were then placed on a germination medium containing 0.6% agar-solidified MS basal medium (Murashige and Skoog (1962) A revised medium for rapid growth and bioassays with tobacco callus cultures. Physiol Plant 15:473-479) and 2% sucrose. The pH was maintained at 5.8. The petri plates were placed in a room at 37° C. for overnight growth or imbibition of seeds. The seed coat was removed, followed by removing part of the hypocotyl, keeping about 0.5 cm of the hypocotyl. One cotyledon was removed along with its adjacent axillary shoot bud and was discarded. On the remaining cotyledon, the primary leaves were broken apart using a scalpel, leaving the primary leaf bases on the epicotyl. (FIG. 4)

Agrobacterium strain (LBA 4404). containing the plasmid pNOV 2145 (ZsGreen1and PMI, as described in Example 1) was streaked from frozen glycerol stocks onto YEP plates (yeast extract 10 g/L, peptone 5 g/L, NaCl 5g/L, bacto agar 15 g/L) containing appropriate antibiotic (100 mg/L spectinomycin). Agrobacterium was then incubated at 27° C. for 1-2 days. A scoop of Agrobacterium from plates were grown on 100 ml YEP liquid medium containing an antibiotic (100 mg/L spectinomycin) for overnight growth at 27° .C on a shaker. Bacterial suspensions were centrifuged at about 1500 g for 15 minutes and resuspended to a density of OD₆₆₀=0.2 or 0.65 in a co-cultivation liquid medium (B₅ salts 0.05X (Sigma), B₅ vitamins: (0.05X) (B₅ vitamin composition (1X): inositol 100 mg/L, nicotinic acid 1 mg/L, pyridoxine HCl 1 mg/L, thiamine HCl 10 mg/L), acetosyringone 40 mg/L, sucrose 20 g/L, IBAP 2 mg/L, GA₃ 0.25 mg/L, MES (Morpholino ethanesulfonic acid) 3.9 g/L, and pH 5.4.

The explants containing the target tissue were immersed into Agrobacterium suspension and incubated for 1-2 hours. The Agrobacterium suspension was poured off, and the treated explants were placed onto a filter paper inside co-cultivation plates. The adaxial side of the explants was kept in contact with the filter paper. The co-cultivation solid medium was composed of B₅ salts (Sigma, 0.05X), B₅ vitamins (0.05X), 40 mg/L acetosyringone, sucrose 20 g/L, BAP 2 mg/L, GA₃ 0.25 mg/L, MES 3.9 g/L, and pH 5.4. The medium was solidified with 0.5% purified agar (Sigma).

The explants were co-cultivated with the Agrobacterium at 20-23° C. for a period of 2-5 days, under 16 h light/8 h dark conditions. After co-cultivation, the explants were washed in sterile water containing 250 mg/L cefotaxime, primary and secondary meristems were removed, and the explants were transferred to regeneration medium (i.e., REG-1 medium). During the regeneration process, any axillary shoots adjacent to the cotyledon were also removed to encourage growth from the area of the primary leaf base.

REG-1 medium contained MS salts (1X), B₅ vitamins (1X), KNO₃ 1 g/L, BAP 1 mg/L, ticarcillin 300 mg/L, cefotaxime 100 mg/L, glutamine 250 mg/L, asparagine 50 mg/L, mannose 15-30 g/L, sucrose 0, 0.25, and 1 g/L, pH 5.6, and purified agar 10 g/L. Five explants were placed in each petri plate in an upright position, such that the epicotyl end of the explant was inserted into the medium. The plates were kept inside a plastic container and placed in a culture room at 22-25° C., under an 18-20 hr light/4-6 hr dark cycle at 60-100 μE m⁻² S⁻¹. After 2 weeks on REG-1 medium, explants were transferred to REG-2 medium, which contained MS salts (1X) and B₅ vitamins (1X), KNO₃ 1 g/L, BAP 0.5 mg/L, ticarcillin 300 mg/L, cefotaxime 100 mg/L, glutamine 250 mg/L, asparagine 50 mg/L, mannose 15 g/L, and sucrose 1 g/L. The media pH was maintained at 5.6, and the media was solidified with purified agar 10 g/L.

At 4-6 weeks, the soybean cultures were transferred to REG-3 medium for continuing selection and shoot development. REG-3 medium contained MS salts (1X), B₅ vitamins (1X), KNO₃ 1 g/L, BAP 0.2 mg/L, GA₃ 0.5 mg/L, IBA 0.1 mg/L, ticarcillin 300 mg/L, cefotaxime 100 mg/L, glutamine 250 mg/L, asparagine 50 mg/L, mannose 15 g/L, sucrose 1 g/L, pH 5.6, and the medium was solidified with purified agar 10 g/L. Dcad tissue was removed and explants with regenerating shoots were subcultured in fresh REG-3 medium every two weeks. Elongated shoots were continuously harvested from the cultures when they reached about 2-4 cm in length. At that time, shoots were transferred to a rooting medium, which contained MS salts (0.5X), B₅ Vitamins (0.5X), glutainine 250 mg/L, asparagine 50 mg/L, KNO₃ 1 g/L, cefotaxime 100 mg/L, ticarcillin 300 mg/L, sucrose 15 g/L, IBA 0.5 mg/L, pH 5.6, and purified agar 10 g/L.

Rooted transgenic shoots expressing a fluorescent protein gene (ZsGreen1) were transferred to 2″ pots which contained moistened Fafard germinating mix (Conrad Fafard Inc., MA, USA) and were kept covered with plastic cups for maintaining moisture for approximately 2 weeks. Plants were acclimatized at 27-29° C. day temperature, 21° C. night temperature, and a 16h photoperiod (20-40 μE m⁻² S⁻¹ light intensity). When new leaves began to emerge, plants were transferred to one-gallon pots which contained a soil mixture composed of 50-55% composted pine bark, 40-45% Peat, 5-10% Perlite (Sungrow Horticultural Supply, Pine Bluff, Ark.). Acclimatized soybean plants were grown in the greenhouse at 27-29° C. day temp, 21° C. night temp, 400-600 μE m⁻² S⁻¹ light intensity, 70-95% relative humidity, and a 16 hr photoperiod. The plants were fertilized with osmocote (Scotts-Sierra Horticultural Products Company, Ohio; 17-6-12) twice (5-8 g/gallon soil) during the growth period. Transformation was confirmed by Taqman analysis for the presence of the fluorescent protein gene as well as the PMI gene in the leaves of the greenhouse grown plants. Expression of the fluorescent protein gene in the transformed soybean tissue was also confirmed by visualizing the expression using a fluorescent microscope.

Six transgenic plants developed using the gene construct pNOV2145 were confirmed by Southern blot analyses. Progeny analysis of one event for either the PMI gene or the ZsGreen1 gene revealed one integration site of the T-DNA into the genome of the transformed soybean, and the progeny segregated in a 3:1 ratio in the T1 generation.

TABLE 1
Transformed shoots expressing fluorescent
protein gene (Zsgreen1)

| | | Transformed | |
|---|---|---|---|
| Expt No. | Gene construct | shoots/explant | Percent tr. shoots |
| 75 | pNOV2145 | 5/45 | 11 |
| 89 | pNOV2145 | 5/75 | 7 |

### EXAMPLE 3

Soybean seeds (Var. S42 H1) were surface sterilized and explants were prepared as described in Example 2.

Agrobacterium strain (LBA 4404) carrying the plasmid pNOV2147 was prepared as described in Example 1. The final bacterial concentration was adjusted to OD ₆₆₀=0.60 with a co-cultivation liquid medium. The conditions for explant preparation, Agrobacterium inoculation, and co-cultivation were the same as those described in Example 2.

Following three days of co-cultivation in a solid co-cultivation medium, excessive Agrobacterium was washed off, primary and secondary meristems were removed, and the explants were transferred to REG-1 medium. They were cultured at 28-30° C. in 16h light and 8h dark conditions. After 2 weeks on REG-1 medium, the cultures were transferred to REG-2 medium. During this regeneration process, only shoots arising from the base of a primary leaf were kept. At about the 4th week, the shoot cultures were transferred to REG-3 medium. They were then transferred to fresh REG-3 medium every 10-14 days. As in REG-1 and REG-2 medium, only the new shoots arising from the base of a primary leaf were kept while the rest of the shoots were removed. When elongated shoots reached about 24 cm in length, they were separated from the rest of the shoot cultures and transferred to rooting medium.

Five transgenic shoots out of 35 explants were identified as expressing the cyano fluorescent protein gene (Table 2).

TABLE 2
Transformed shoots expressing the cyano fluorescent protein gene

| Experiment | | Transformed | |
|---|---|---|---|
| No. | Gene construct | shoots/explants | % transformed shoots |
| 92 | pNOV2147 | 5/35 | 14 |

### EXAMPLE 4

Mannose Treatment During Co-cultivation

The gene construct used in this example was pNOV2145 (which comprises ZsGreen1 and PMI genes, as described in Example 1). The procedures for preparing the explants, Agrobacteria suspensions, and inoculation of explants with bacterial suspensions were carried out as described in Example 2. The final bacterial concentration was adjusted to OD ₆₆₀=0.55 or 0.85.

Following the inoculation step, explants were transferred to a co-cultivation medium containing either 20 g/L sucrose or 15 g/L mannose and were kept at 20-23° C. under 16h light and 8h dark conditions.

After 3-5 days of co-cultivation, expression of the fluorescent protein gene was visualized using a fluorescent microscope. Explants that were inoculated with Agrobacterium in a mannose-containing co-cultivation medium showed at least two-fold the number of fluorescent spots compared to those co-cultivated in a sucrose-containing co-cultivation medium. Subsequent shoot regeneration and selection steps were followed as those described in Example 2.

A significant increase in the production of transformed shoots was observed in the experiments where mannose was included in the co-cultivation medium (Table 3). Five transformed shoots from co-cultivation medium that included mannose were rooted and transferred to soil. Subsequent analysis by Taqman as well as Southern blot confirmed the integration of the transgenes. Transgene expression in the T1 progeny confirmed the germline transmission of the transgenes.

TABLE 3
Transformed shoots expressing ZsGreen1 fluorescent
protein gene where explants and Agrobacteria were
co-cultivated in mannose or sucrose

| Experi- | | | Transformed | % |
|---|---|---|---|---|
| ment | Gene | Co-culture in | shoots/ | Transfor- |
| No. | construct | mannose/sucrose | explants | mation |
| 87 | pNOV2145 | Sucrose | 0/60 | 0 |
| | | Mannose | 6/80 | 7.5 |
| 102 | pNOV2145 | Sucrose | 1/20 | 5 |
| | | Mannose | 8/40 | 20 |

### EXAMPLE 5

In this example, Agrobacterium EHA101 comprising the plasmid pNOV2105 (SMAS-PMI SMAS-GUS, as described in Example 1) was used in soybean transformation. The preparation of the explants, Agrobacteria suspension, and inoculation of explants with Agrobacteria were the same as those described in Example 2. The final bacterial concentration was adjusted to OD ₆₆₀=0.45 or 0.6.

Following Agrobacterium inoculation, explants were transferred to a co-cultivation medium containing either 20 g/L sucrose or 15 g/L mannose. Co-cultivation was carried out at 20-23° C. under a 16h light and 8h dark conditions. Following 3-5 days of co-cultivation, GUS gene expression was visualized using a histochemical gus assay. Explants co-cultivated in mannose-containing co-cultivation medium showed at least two-fold the number of GUS spots compared to those co-cultivated in sucrose-containing co-cultivation medium. Shoot regeneration and selection were carried out as described in Example 2. A significant increase in the production of transformed shoots was observed in the experiment in which mannose was added into the co-cultivation medium. (Table 4).

TABLE 4
Transformed shoots expressing GUS gene

| Experi- | | | Transformed | % |
|---|---|---|---|---|
| ment | Gene | Co-cultivation in | shoots/ | Transfor- |
| No. | construct | mannose/sucrose | explant | mation |
| 63 | pNOV2105 | sucrose | 5/60 | 8 |
| 81 | pNOV2105 | Sucrose | 2/30 | 7 |
| | | Mannose | 5/30 | 17 |

### EXAMPLE 6

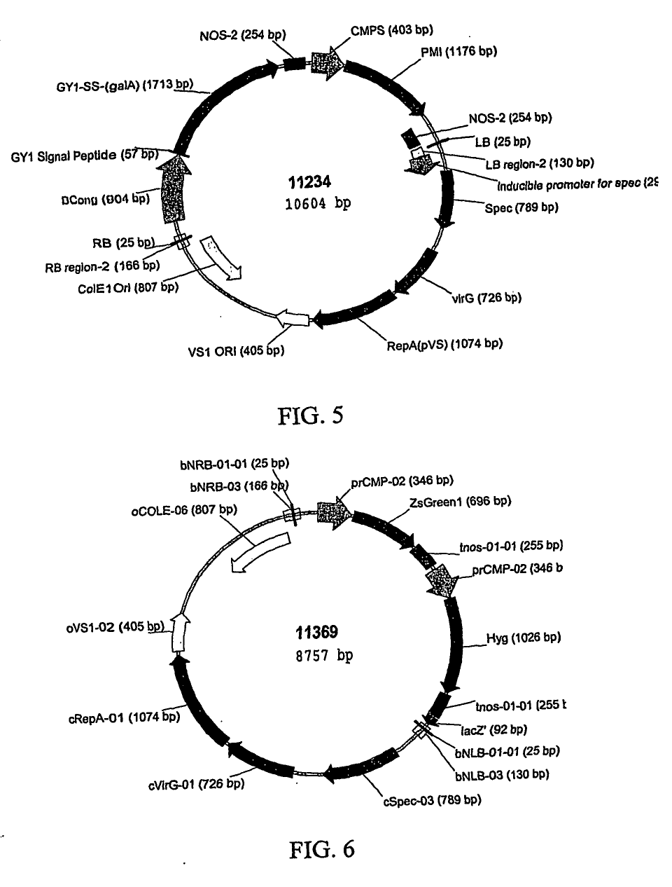

In this example, Agrobacterium EHA101 comprising the plasmid pBSC11234 (FIG. 5) was used in soybean transformation. The components and sequence of pBSC11234 are set forth in SEQ ID NO:3. pBSC11234 comprises a CMP-PMI : beta conglycinin-galactosidase gene construct. The preparation of the explants, Agrobacteria suspension, and inoculation of explants with Agrobacteria were the same as those described in Example 2. The final bacterial concentration was adjusted to OD ₆₆₀=0.6. The co-cultivation liquid medium contained B₅ salts (0.1X), B₅ vitamins (1X), acetosyringone 80 mg/L, sucrose 20 g/L, BAP 2 mg/L, GA₃ 0.25 mg/L, MES 3.9 g/L, and pH 5.4. Solid co-cultivation medium was prepared by incorporating 5 g/L purified agar to the liquid co-cultivation medium.

Following Agrobacterium inoculation, explants were transferred to a solid co-cultivation medium and cultured at 20-24° C. under 16h light and 8h dark conditions. Following 3-5 days of co-cultivation, primary and secondary shoot meristems were removed and discarded, and the resulting explants were transferred to REG-4 medium, which contained B₅ salts (1X), B₅ Vitamins (1X), BAP 1 mg/L, glutamine 50 mg/L, asparagine 50 mg/L, cefotaxime 100 mg/L, ticarcillin 300 mg/L, mannose 15-20 g/L, sucrose 0, 0.25, or 1 g/L, purified agar 10 g/L, and pH at 5.6. After a period of 5-7 days, any shoot grown from the axillary meristem close to the cotyledon was removed, and the explants were transferred to REG-5 medium, which contained B₅ salts (1X), B₅ Vitamins (1X), BAP 0.5 mg/L, glutamine 50 mg/L, asparagine 50 mg/L, cefotaxime 100 mg/L, ticarcillin 300 mg/L, mannose 15 g/L, sucrose 1 g/L, purified agar 10 g/L, and pH at 5.6. At four weeks, explants were transferred to REG-6 medium for elongation of shoots. REG-6 medium contained MS salts (1X), MS Vitamins (1X) (MS vitamin composition: inositol 100 mg/L, nicotinic acid 0.5 mg/L, pyridoxine HCl 0.5 mg/L, thiamine HCl 0.1 mg/L, glycine 2 mg/L), myo-inositol 200 mg/L, BAP 0.2 mg/L, zeatin riboside 0.5 mg/L, IBA 0.1 mg/L, GA₃ 1 mg/L, glutamine 50 mg/L, asparagine 50 mg/L, ticarcillin 300 mg/L, mannose 15 g/L, sucrose 5 g/L, silver nitrate 0.8 mg/L, purified agar 10 g/L, and pH 5.6. Explants were transferred to fresh REG-6 medium every two weeks. Elongated shoots (24 cm long) were removed and rooted in rooting medium and transferred to soil. The rooting medium contained MS salts (1X), B₅ Vitamins (1X), glutamine 100 mg/L, asparagine 100 mg/L, IBA 0.7 mg/L, timentin 100 mg/L, and sucrose 15 g/L. Taqman analysis confirmed the presence of the transgenes (alpha galactosidase and phosphomannose isomerase) in leaf samples from two events.

### EXAMPLE 7

In this example, Agrobacterium EHA101 comprising the plasmid pBSC11369 (FIG. 6) was used in soybean transformation. The components and sequence of pBSC11369 are set forth in SEQ ID NO:4. pBSC11369 comprises a CMP-HPT: CMP-ZsGreen1 gene construct. The preparation of the explants, Agrobacteria suspension, and inoculation of explants with Agrobacteria were the same as those described in Example 2. The final bacterial concentration was adjusted to OD ₆₆₀=0.6. The co-cultivation liquid medium contained B₅ salts (0.1X), B₅ vitamins (1X), acetosyringone 80 mg/L, sucrose 20 g/L, BAP 2 mg/L, GA₃ 0.25 mg/L, MES 3.9 g/L, and pH 5.4. Solid co-cultivation medium was prepared by incorporating 5 g/L purified agar to the liquid co-cultivation medium.

Following Agrobacterium inoculation, explants wore transferred to a solid co-cultivation medium and cultured at 20-24° C. under 16h light and 8h dark conditions. Following 3-5 days of co-cultivation, explants were transferred to REG-7 medium after removing primary and secondary meristems from the explants in order to encourage shoot growth from the primary leaf base area REG-7 medium contained B₅ salts (1X), B₅ Vitamins (1X), BAP 1 mg/L, glutamine 50 mg/L, asparagine 50 mg/L, cefotaxime 100 mg/L, ticarcillin 300 mg/L, sucrose 30 g/L, hygromycin 2-5 mg/L, purified agar 10 g/L, and pH 5.6. Explants were placed in an upright position such that the epicotyl end of the explant was inserted into the medium. After a period of 7-10 days, any shoots grown from the axillary meristem close to the cotyledon were removed. Explants were transferred to fresh REG-8 medium, which contained B₅ salts (1X), B₅ Vitamins (1X), BAP 0.5 mg/L, glutamine 50 mg/L, asparagine 50 mg/L, cefotaxime 100 mg/L, ticarcillin 300 mg/L, sucrose 30 g/L, purified agar 10 g/L, and pH at 5.6. After another two weeks, explants were transferred to REG-9 medium and subcultured thereafter every two weeks. REG-9 medium contained MS salts (1X), MS Vitamins (1X), myo-inositol 200 mg/L, BAP 0.2 mg/L, zeatin riboside 0.5 mg/L, IBA 0.1 mg/L, GA₃ 1 mg/L, glutamine 50 mg/L, asparagine 50 mg/L, silver nitrate 0.8 mg/L, ticarcillin 300 mg/L, sucrose 30 g/L, hygromycin 0.1-0.2 mg/L, purified agar 10 g/L, and pH 5.6. Elongated shoots (24 cm long) were removed, rooted in rooting medium, and then transferred to soil. The rooting medium contained MS salts (1X), B₅ Vitamins (1X), glutamine 100 mg/L, asparagine 100 mg/L, IBA 0.7 mg/L, timentin 100 mg/L, and sucrose 15 g/L. Taqman analysis confirmed the presence of the transgenes (HPT as well as ZsGreen1) in leaf samples obtained from five events. Expression of the ZsGreen1 gene in plant parts was confirmed by visualization under a fluorescent microscope.

All publications, patents, and patent applications cited herein are incorporated by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method for transforming soybean cells or tissue, comprising:
(a) preparing an explant from a soybean seed by.
(i) removing a hypocotyl from said soybean seed;
(ii) removing one cotyledon along with its adjacent axillary bud, leaving primary leaves attached to a remaining cotyledon; and
(iii) removing a portion of a primary leaf from said remaining cotyledon, thereby generating a primary leaf base; and
(b) co-cultivating said explant with Agrobacterium comprising at least one nucleic acid of interest to be incorporated into a genome of one or more soybean cells.

2. The method of claim 1, further comprising cultivating at least one formed shoot in a medium containing a selection agent.

3. The method of claim 2, wherein said at least one nucleic acid of interest comprises a selectable marker gene.

4. The method of claim 3, wherein said selectable marker gene is a phosphomannose isomerase gene.

5. The method of claim 4, wherein said selection agent is mannose.

6. The method of claim 4, wherein co-cultivation with said Agrobacterium is carried out in the presence of mannose.

7. The method of claim 2, further comprising inducing shoot formation from said primary leaf base.

8. The method of claim 7, wherein shoot formation is induced by culturing said primary leaf base in a medium comprising a shoot-inducing hormone.

9. The method of claim 8, wherein said shoot-inducing hormone comprises at least one of an auxin, a cytokinin, and a gibberellic acid.

10. The method of claim 9, wherein said auxin is selected from the group consisting of IAA, NAA, and IBA.

11. The method of claim 9, wherein said cytokinin is selected from the group consisting of benzylaminopurine (BAP), thidiazuron, kinetin, and isopentenyl adenine.

12. The method of claim 7, wherein induction of shoot formation comprises removing one or more of a primary meristem, a secondary meristem, and an axillary meristem attached to a cotyledon.

13. The method of claim 7, further comprising selecting a transformed shoot.

14. The method of claim 13, further comprising regenerating a selected transformed shoot into a soybean plant.

15. The method of claim 1, wherein said soybean seed is a mature seed.

16. The method of claim 1, wherein said soybean seed is an immature seed.

17. The method of claim 1, wherein said soybean seed is a germinated seed.

18. A method for producing a stably transformed soybean plant, comprising:
(a) preparing an explant from a soybean seed by:
(i) removing a hypocotyl from said soybean seed;
(ii) removing one cotyledon along with its adjacent axillary bud, leaving primary leaves attached to a remaining cotyledon; and
(iii) removing a portion of each primary leaf from said remaining cotyledon, thereby generating a pair of primary leaf bases;
(b) co-cultivating said explant with Agrobacterium comprising a nucleic acid of interest to be incorporated into a genome of a soybean cell;
(c) inducing shoot formation from each primary leaf base;
(d) cultivating at least one formed shoot in a medium containing a selection agent;
(e) selecting a transformed shoot; and
(f) regenerating a selected transformed shoot into a soybean plant.

19. A transgenic soybean plant regenerated from soybean cells or tissue transformed according to the method of claim 1.

20. A transgenic seed produced by the transgenic plant of claim 19.

21. A transgenic soybean plant regenerated from soybean cells or tissue transformed according to the method of claim 18.

22. A transgenic seed produced by the transgenic plant of claim 21.
